(19) United States
(12) Patent Application Publication
(10) Pub. No.: US 2022/0108803 A1
(43) Pub. Date: Apr. 7, 2022

(54) METHOD FOR COVID-19 DETECTION TO SPREAD PREVENTION AND MEDICAL ASSISTANCE USING MACHINE LEARNING

(71) Applicants: Gaurav Indra, Delhi (IN); Romany F. Mansour, El-Kharga (EG); B.K. Sarkar, NAVI MUMBAI (IN); Maha M. Althobaiti, Taif (SA); Sadhana Dubey, Shenzhen (CN); Amit Kumar Tyagi, Chennai (IN); Vineet Tirth, Abha (SA); Neeraj Kumar Shukla, Abha (SA); Munawira Kotyad, NAVI MUMBAI (IN); LATA MENON, NAVI MUMBAI (IN)

(72) Inventors: Gaurav Indra, Delhi (IN); Romany F. Mansour, El-Kharga (EG); B.K. Sarkar, NAVI MUMBAI (IN); Maha M. Althobaiti, Taif (SA); Sadhana Dubey, Shenzhen (CN); Amit Kumar Tyagi, Chennai (IN); Vineet Tirth, Abha (SA); Neeraj Kumar Shukla, Abha (SA); Munawira Kotyad, NAVI MUMBAI (IN); LATA MENON, NAVI MUMBAI (IN)

(21) Appl. No.: 17/551,376

(22) Filed: Dec. 15, 2021

(51) Int. Cl. G16H 50/20; G06T 5/00; G06T 7/11; G06T 7/00; G06V 40/16; G06T 7/187; A61B 6/00; A61B 5/01; A61B 5/00; G16H 30/20

(52) U.S. Cl. CPC G16H 50/20; G06T 2207/10081; G06T 7/11; G06T 7/0014; G06V 40/161; G06T 7/187; A61B 6/5205; A61B 6/5258; A61B 5/015; A61B 5/7475; A61B 5/7465; A61B 5/746; G16H 30/20; G06T 2207/20084; G06T 5/002

(57) ABSTRACT

The present disclosure generally relates to a method for COVID-19 detection for spread prevention and medical assistance using machine learning comprises pre-processing a CT image set for removing noise using HU values; performing a region-based segmentation using a three-dimensional convolutional neural network model; classifying each segmented region by employing a classification model; and comparing classified segmented region with a dataset for detecting COVID-19 using a machine learning approach thereby generating an alert about the COVID-19 positive person.

### FIELD OF THE DISCLOSURE

The present disclosure relates to a method for COVID-19 detection to spread prevention and medical assistance using machine learning. In more details, the method facilitates in detecting COVID-19 and ensuring social distancing.

### BACKGROUND

Coronavirus infection is airborne and can without much of a stretch spread and contaminate individuals. As per the Centers for Disease Control and Prevention (CDC), the tainted individuals show a scope of indications like dry hack, windedness, weariness, losing the feeling of taste and smell, the runs, and clog. Tainted patients can likewise introduce fever scenes. Surprisingly, a few patients who have gotten the infection probably won't show any of the previously mentioned side effects. They can feel totally ordinary conveying the infection and proceeding to spread the illness without knowing.

As COVID-19 has a quick nature of spreading, the World Health Organization (WHO) proclaimed it as a worldwide pandemic in March 2020. At the hour of composing this disclosure (i.e., September 2020), the absolute number of affirmed COVID-19 cases overall was more than 32 million. To handle this episode, researchers in various examination networks are looking for a wide assortment of PC helped frameworks like the Internet of Things Machine Learning (ML) or Deep Learning (DL) procedures.

There were two scourges in the past from the Covid family including Severe Acute Respiratory Syndrome (SARSCoV) and Middle Eastern Respiratory Syndrome (MERS). SARS-CoV is a respiratory infection that was contagious from one individual to another and it was first distinguished in 2003. The infection had more than 8,000 affirmed cases overall during its course which influenced more than 26 nations MERS is likewise a respiratory infection with comparable side effects of SARS-CoV. ML, as a subset of Artificial Intelligence (AI), has shown a great deal of possibilities in numerous ventures.

ML procedures can be customized to emulate human insight. For instance, in the medical services industry, ML procedures can be prepared and utilized towards clinical finding. ML models have been unfathomably prepared over a dataset comprising of clinical pictures like Computed Tomography (CT) Scan, Magnetic Resonance Imaging (MRI), peculiarities. During past pandemics, ML procedures have been broadly carried out to help medical care experts for better activities in regards to the illnesses. For instance, a ML model that uses GPS innovation alongside distributed computing force and Google Maps to address conceivably contaminated patients and give an elective course to uninfected clients coming about in possibly moderating the spread. The model arrives at the characterization exactness of 80% in re-directing away from tainted patients.

This sort of inside and out investigation can quickly screen the public response. It can likewise help policymakers in making the right moves in diminishing trepidation and pain from general society in regards to MERS. ML has additionally been broadly utilized to further develop clinical dynamic in regards to the current COVID-19 pandemic. Specialists, utilizing ML calculations and grouping strategy, can figure the spread in regions. ML techniques for picture characterization are utilized by established researchers to help in diagnosing the dangerous infection. With the goal of discovering a remedy for the infection, ML calculations are utilized to assess how reliable are off-the-counter medications might be utilized to help tainted patients.

The first Covids found on the planet were liable for respiratory and intestinal contaminations, of which by far most had a self-restricted course and drove generally to manifestations of normal virus. Be that as it may, they can ultimately form into genuine contaminations in bunches in danger (heart infections, diabetes, among others), in the old and furthermore in kids. Prior to the start of this current pandemic, two profoundly pathogenic Covid species (SARS and MERS) were portrayed and were liable for flare-ups of extreme intense respiratory conditions.

Concerning new (COVID-19) it was perceived as a causative specialist of pneumonia that prompts extreme intense respiratory disorder (SARS-CoV-2). One of its principle challenges is its quick bandwidth and, at times, movement to serious pneumonic conditions that have requested from the wellbeing framework a consideration and battle technique never found in the entire world. In Brazil, the assumption is to be of dramatic development, which is the reason there is a need to execute extreme measures to control populace course and counteraction. Because of the speed of transmission, in many nations, including Brazil, beginning phase preventive measures were not carried out, causing a blast of indicative cases, a large number of them serious, with delayed interest from tertiary wellbeing administrations.

Given this situation, the inescapable rise of an enormous unexpected of basically sick patients with COVID-19, with various guesses, made it essential to look for early demonstrative systems for better screening and therapy sufficiency for each situation. In this unique circumstance, even before affirmation of the contamination, evaluating for patients with respiratory indications is brought out through clinical examination and imaging tests like Chest Computed Tomography (CT).

In clinical assessment, the principle depicted manifestations of contamination are fever (88.5%), hack (68.6%), myalgia or weakness (35.8%), sputum (28, 2%) and dyspnea (21.9%). Different indications likewise depicted incorporate cerebral pain and tipsiness (12.1%), loose bowels (4.8%), queasiness and spewing (3.9%). Furthermore, some haematological changes were noticed: lymphocytopenia (64.5%), expanded C-responsive protein (CRP) (44.3%), expanded lactic dehydrogenase (DHL) (28.3%), and leukopenia (29, 4%). Chest CT is thinking about as the best imaging technique for appraisal of COVID-19, since customary radiography has low affectability, outstandingly in beginning phases. Regular discoveries portrayed in the writing incorporate ground-glass opacities (GGO) with a more fringe appropriation, related with septal thickening and solidifications, generally influencing various projections, albeit these discoveries can likewise be found in other viral pneumonias.

Along these lines, AI applied to symptomatic imaging can empower the improvement of apparatuses that can normalize the determination and give potential discoveries reminiscent of the presence of the infection, its seriousness, and accordingly its guess. Since the start of the pandemic, because of its crisis, a few examinations opened up to attempt to foresee most noticeably terrible results. Fundamental conceivable danger factors were assessed in review examines. A large portion of these investigations show old age, weight and other comorbidities (diabetes, serious asthma and other respiratory sicknesses, heart, liver, neurological and kidney infections and immune system illnesses) as the principle players for a most noticeably terrible result.

AI has been utilized by a developing number of studies in this situation and in other wellbeing related fields, going from assisting with analysis until giving more vigorous proof to asset distribution upheld that further examinations are need to show all the capability of this apparatus in clinical practice.

In the view of the forgoing discussion, it is clearly portrayed that there is a need to have a method for COVID-19 detection to spread prevention and medical assistance using machine learning.

### BRIEF SUMMARY OF THE DISCLOSURE

The present disclosure seeks to provide a method for COVID-19 detection for controlling the spread of the infection, distinguishing the infection, or in any event, planning and assembling an immunization or medication to battle it.

In an embodiment, a method for COVID-19 detection to spread prevention and medical assistance using machine learning is disclosed. The method includes pre-processing a CT image set for removing noise using HU values. The system further includes performing a region-based segmentation using a three-dimensional convolutional neural network model. The system further includes classifying each segmented region by employing a classification model. The system further includes comparing classified segmented region with a dataset for detecting COVID-19 using a machine learning approach thereby generating an alert about the COVID-19 positive person.

In another embodiment, social distancing and mask detection is performed on the basis of thermal images using a thermal imagining camera.

In another embodiment, a warm screening gadget is configured with the thermal imagining camera for interpreting infrared warm imaging and AI face acknowledgment for separating swarm structures or doorways more effective, wherein the gadget catches a guest's temperature and furthermore checks whether the person in question is wearing a face veil or not.

In another embodiment, a chatbot is executed to provide an online discussion to a client about one or the other content or voice shows on a user interface, wherein the user interface is installed on a user computing device.

In another embodiment, the chatbot promotes assisting the clients with a superior comprehension of their circumstance and gives a few clues to clients so the person is allowed to make legitimate strides, wherein the chatbot suggests that an individual having a positive COVID-19 test along with a person in close contact with the affirmed cases is suggested to be isolated for 14 days.

In another embodiment, one of an unmanned aerial vehicle (UAV) or a robot with the machine learning approach in light of the requirement for keeping social separation in swarms.

In another embodiment, the generated alert is shown in the user interface of the computing device so that any of the person having the user interface gets alert about the COVID-19.

In another embodiment, the alert is automatically generated and sent to registered computing device and the user interface.

In another embodiment, the region-grown operator performs connected-components analysis on gray-scale pixel values and the neighboring pixels are compared to a reference pixel value in each pixel of the image for the region-based segmentation.

In another embodiment, an individual is screened with a live stream on a dashboard which permits cautioning the experts if there should arise an occurrence of breaking of COVID-19 guidelines.

An object of the present disclosure is to provide machine learning based detection of COVID-19.

Another object of the present disclosure is to promote maintaining of social distancing and wearing of face mask to avoid COVID-19 transmission.

Another object of the present disclosure is to provide a assess potential changes in Chest CT, through a score, that propose a more terrible anticipation in patients with COVID-19, and to distinguish designs corresponded with more awful clinical turns of events, to direct, in the forthcoming unfurling of the investigation, the assessment of prognostic markers emerging computerized examinations of Chest CT and add to focusing on treatment as indicated by seriousness (orotracheal intubation, hospitalization).

Another object of the present disclosure is to provide a information base with clinical pictures and their individual anonymized reports for CT methodology, in various transform utilitarian changes, in patients with intense respiratory disorders.

Another object of the present disclosure is to provide a Evaluate the exhibition of AI calculations in this information for undertakings like grouping, division, picture enrollment and understanding of reports.

Another object of the present disclosure is to provide a Evaluate the effect of the utilization of these models on clinical act of imaging experts.

Yet another object of the present disclosure is to deliver an expeditious and cost-effective method for COVID-19 detection to spread prevention and medical assistance using machine learning.

To further clarify advantages and features of the present disclosure, a more particular description of the disclosure will be rendered by reference to specific embodiments thereof, which is illustrated in the appended drawings. It is appreciated that these drawings depict only typical embodiments of the disclosure and are therefore not to be considered limiting of its scope. The disclosure will be described and explained with additional specificity and detail with the accompanying drawings.

### BRIEF DESCRIPTION OF FIGURES

These and other features, aspects, and advantages of the present disclosure will become better understood when the following detailed description is read with reference to the accompanying drawings in which like characters represent like parts throughout the drawings, wherein:

FIG. 1 illustrates a flow chart of a method for COVID-19 detection to spread prevention and medical assistance using machine learning in accordance with an embodiment of the present disclosure; and

FIG. 2 illustrates a block diagram of a system for COVID-19 detection to spread prevention and medical assistance using machine learning in accordance with an embodiment of the present disclosure.

Further, skilled artisans will appreciate that elements in the drawings are illustrated for simplicity and may not have necessarily been drawn to scale. For example, the flow charts illustrate the method in terms of the most prominent steps involved to help to improve understanding of aspects of the present disclosure. Furthermore, in terms of the construction of the device, one or more components of the device may have been represented in the drawings by conventional symbols, and the drawings may show only those specific details that are pertinent to understanding the embodiments of the present disclosure so as not to obscure the drawings with details that will be readily apparent to those of ordinary skill in the art having benefit of the description herein.

### DETAILED DESCRIPTION

For the purpose of promoting an understanding of the principles of the disclosure, reference will now be made to the embodiment illustrated in the drawings and specific language will be used to describe the same. It will nevertheless be understood that no limitation of the scope of the disclosure is thereby intended, such alterations and further modifications in the illustrated system, and such further applications of the principles of the disclosure as illustrated therein being contemplated as would normally occur to one skilled in the art to which the disclosure relates.

It will be understood by those skilled in the art that the foregoing general description and the following detailed description are exemplary and explanatory of the disclosure and are not intended to be restrictive thereof.

Reference throughout this specification to “an aspect”, “another aspect” or similar language means that a particular feature, structure, or characteristic described in connection with the embodiment is included in at least one embodiment of the present disclosure. Thus, appearances of the phrase “in an embodiment”, “in another embodiment” and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components.

Unless otherwise defined, all technical and scientific terms used herein have the same meaning as commonly understood by one of ordinary skill in the art to which this disclosure belongs. The system, methods, and examples provided herein are illustrative only and not intended to be limiting.

Embodiments of the present disclosure will be described below in detail with reference to the accompanying drawings.

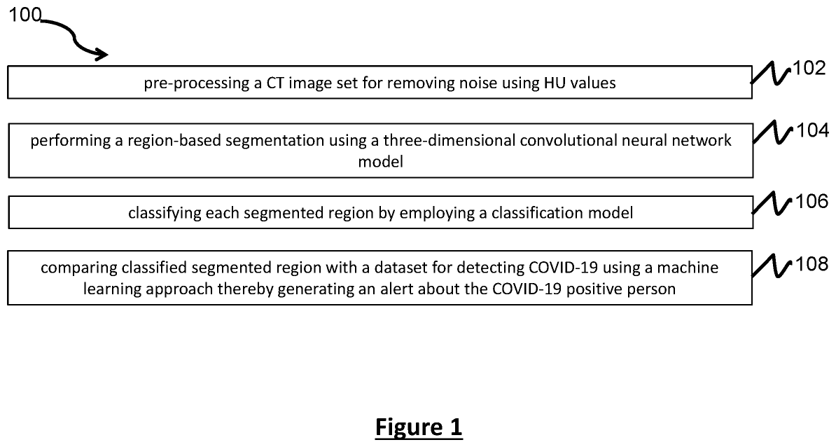

Referring to FIG. 1, a flow chart of a method for COVID-19 detection to spread prevention and medical assistance using machine learning is illustrated in accordance with an embodiment of the present disclosure. At step 102, the method 100 includes pre-processing a CT image set for removing noise using HU values.

At step 104, the method 100 includes performing a region-based segmentation using a three-dimensional convolutional neural network model.

At step 106, the method 100 includes classifying each segmented region by employing a classification model.

At step 108, the method 100 includes comparing classified segmented region with a dataset for detecting COVID-19 using a machine learning approach thereby generating an alert about the COVID-19 positive person.

In another embodiment, social distancing and mask detection is performed on the basis of thermal images using a thermal imagining camera.

In another embodiment, a warm screening gadget is configured with the thermal imagining camera for interpreting infrared warm imaging and AI face acknowledgment for separating swarm structures or doorways more effective, wherein the gadget catches a guest's temperature and furthermore checks whether the person in question is wearing a face veil or not.

In another embodiment, a chatbot 210 is executed to provide an online discussion to a client about one or the other content or voice shows on a user interface, wherein the user interface is installed on a user computing device.

In another embodiment, the chatbot 210 promotes assisting the clients with a superior comprehension of their circumstance and gives a few clues to clients so the person is allowed to make legitimate strides, wherein the chatbot 210 suggests that an individual having a positive COVID-19 test along with a person in close contact with the affirmed cases is suggested to be isolated for 14 days.

In another embodiment, one of an unmanned aerial vehicle (UAV) or a robot with the machine learning approach in light of the requirement for keeping social separation in swarms.

In another embodiment, the generated alert is shown in the user interface of the computing device so that any of the person having the user interface gets alert about the COVID-19.

In another embodiment, the alert is automatically generated and sent to registered computing device and the user interface.

In another embodiment, the region-grown operator performs connected-components analysis on gray-scale pixel values and the neighboring pixels are compared to a reference pixel value in each pixel of the image for the region-based segmentation.

In another embodiment, an individual is screened with a live stream on a dashboard which permits cautioning the experts if there should arise an occurrence of breaking of COVID-19 guidelines.

Regarding the need of rehearsing social removing utilizing IoT gadgets, AI can carry out a mechanized screening utilizing PC vision techniques. An occurrence of such a gadget is RayVision which guarantees social separating and face veil wearing rules are continued in the group. By utilizing the PC vision methods, it can screen individuals with a live stream on its particular dashboard which permits cautioning the experts if there should arise an occurrence of any standard breaking.

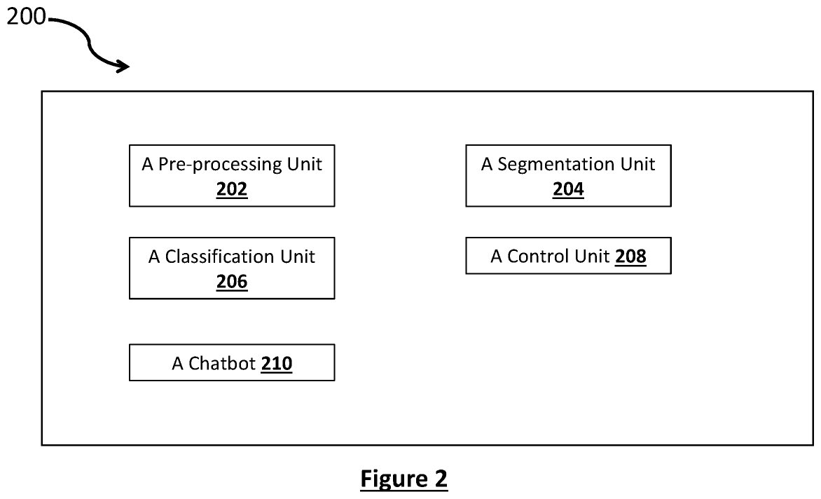

FIG. 2 illustrates a block diagram of a system for COVID-19 detection to spread prevention and medical assistance using machine learning in accordance with an embodiment of the present disclosure. The system 200 includes a pre-processing unit 202 for pre-processing a CT image set for removing noise using HU values. The pre-processing unit 202 is employed to balance light and saturation of the CT image.

In an embodiment, a segmentation unit 204 is connected to the pre-processing unit 202 for performing a region-based segmentation using a three-dimensional convolutional neural network model.

In an embodiment, a classification unit 206 is connected to the segmentation unit 204 for classifying each segmented region by employing a classification model.

In an embodiment, a control unit 208 is connected to the classification unit 206 for comparing classified segmented region with a dataset for detecting COVID-19 using a machine learning approach thereby generating an alert about the COVID-19 positive person.

Diagnosing COVID-19 is quite possibly the main pieces of managing the sickness. Because of low access and high chance of bogus adverse outcomes to the RT-PCR packs, there is a fundamental requirement for utilizing different methodologies, for example, clinical pictures examination for exact and dependable screening and conclusion in COVID-19.

As a general rule, dissecting clinical imaging modalities, for example, chest X-beam and CT-Scan have key commitments in affirming the conclusion of COVID-19 just as screening the movement of the illness. Distinctive ML methods that fuse X-beam and CT-Scan picture preparing approaches could help doctors and medical services experts as a superior way for finding and comprehension of the movement of the COVID-19 illness.

X-beam: During this pandemic, chest imaging can be a significant piece of the COVID-19 in beginning phase of recognition. Arranging patients quickly is what is generally anticipated from these methodologies. Inside the order of clinical imaging, Chest X-Ray (CXR) was prescribed to be executed as the primary clinical imaging in regards to COVID-19 by the Italian Society of Radiology (SIRM.

CXR has an affectability of 67.1% which can be first executed in quite a while incorporating helping radiologists with better COVID-19 cases recognizable proof and quick treatment allocating to the patient. Moreover, CXR is cheap and secure in light of limiting the danger of defilement which makes a more secure working environment for medical services laborers also.

To diminish the measure of work by radiologists, ML procedures can be relegated to arrange patients concerning COVID-19. To do that, specialists are generally centered around the ML arrangement models like Support Vector Machine (SVM), Convolutional Neural Networks (CNN), DL. One methodology executed X-Ray to arrange the lung injuries (brought about by COVID-19) with Multi-Level Threshold (MT) cycle and SVM model.

Initially, the lung picture difference will be improved. Besides, the picture will be diminished into explicit segments (utilizing MT) to keep away from duplication of work on uninfected regions. Ultimately, the SVM model characterizes the areas of the lung concerning the predefined solid lungs. The fostered a stage utilizing an assortment of Deep Convolutional Neural Networks (DCNN) models characterizing inside the SVM with two unique datasets to recognize COVID-19 cases dependent on the connected CRX picture.

The proposed a DCNN model utilizing the information assembled from two emergency clinics in Italy to addresses the significance of AI in the identification of COVID-19. The prepared ML models over an enormous viral pneumonia dataset of CXR pictures to identify inconsistencies. They tried their model on a totally unique dataset that has COVID-19 CXR pictures.

This is done as one of the side effects of COVID-19 can be pneumonia. The outcomes are amazing as the model performs well when tried on the COVID-19 dataset with the Area Under the Curve (AUC) of 83.61%. It is considerably more amazing as the model was prepared on an alternate dataset but performed well. Likewise, used COVIDx dataset, an openly accessible dataset comprises of COVID-19, pneumonia and non-COVID-19 pneumonia related X-beam pictures.

The creators utilized this information to prepare their model for recognition of COVID-19, the Deep Neural Network (DNN) is alluded to as COVID-Net appearance encouraging outcomes in diagnosing contaminated patients. utilized exchange learning approaches like element extraction and calibrating of CNN based models and prepared and tried over comparable datasets accomplishing a forecast exactness up to practically 98%.

They showed that executing move learning can have a huge improvement in outcomes. Most ML classifiers are prepared and tried to accomplish high expectation exactness of COVID-19; in any case, evaluate the vulnerability that could exist by utilizing such classifiers as an essential mechanism of conclusion. A way to deal with approve the ML expectation of conclusion in CXR pictures was evaluated.

It took advantage of a Bayesian Deep Learning classifier to assess the model vulnerability. The outcome examination shows a solid relationship amongst vulnerability and precision of expectation, which implies that the higher the vulnerability result, the more dependable the forecast exactness.

In another embodiment, a chatbot 210 is deployed in the system, which is created to communicate with people by embracing normal dialects are called chatbots. A chatbot 210 fundamentally can speak with various clients and produce appropriate reactions to those client's dependent on their bits of feedbacks. As of late, the COVID-19 pandemic has prompted constructing diverse chatbots as opposed to utilizing hotlines as a specialized technique. This will decrease medical clinic visits and increment the proficiency of conveying.

By and large, chatbots are executed to give an online discussion the client by one or the other content or voice shows on web applications, cell phone applications, channels, and This discussion can assist the client with having a superior comprehension of their circumstance and gives a few clues to clients so the person can make legitimate strides. Chatbots are generally considered as extraordinary compared to other fit to screen patients distantly without com.

The benefits of them incorporate rapidly refreshing data, tediously uplifting new practices like washing hands, and helping with mental help because of the pressure brought about by separation and falsehood. The ML-based chatbots are improved during the preparation methodology and utilizing more information makes this methodology more solid. During the COVID-19 pandemic, chatbots are standing out enough to be noticed to give more insights concerning COVID-19 in various stages.

A wide assortment of chatbots with various dialects have been carried out to help patients at the beginning phase of COVID-19. “Aapka Chkitsak”, an AI-based chatbot 210 created by in India, helps patients with far off meeting in regards to their wellbeing data, and medicines.

The elective theory is that the AI model dependent on clinical, radiological and epidemiological information will actually want to foresee the seriousness forecast of patients contaminated with COVID-19. We will play out a multicenter review longitudinal examination to acquire countless cases in a brief timeframe, for better investigation approval.

The comfort test (something like 20 cases for every result) will be gathered in each middle thinking about the consideration and rejection standards. We will assess patients who enter the medical clinic with clinical signs and side effects of intense respiratory condition, from March to May 2020.

In another embodiment, a set of people signs and manifestations of intense respiratory condition, are incorporated with positive epidemiological history for COVID-19, who have played out a chest processed tomography. We will evaluate chest CT of these patients and to connect them with the course of the sickness.

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

1. A method for COVID-19 detection for spread prevention and medical assistance using machine learning, the method comprises:
pre-processing a CT image set for removing noise using HU values;
performing a region-based segmentation using a three-dimensional convolutional neural network model;
classifying each segmented region by employing a classification model; and
comparing classified segmented region with a dataset for detecting COVID-19 using a machine learning approach thereby generating an alert about the COVID-19 positive person.

2. The method of claim 1, wherein social distancing and mask detection is performed on the basis of thermal images using a thermal imagining camera.

3. The method of claim 1, wherein a warm screening gadget is configured with the thermal imagining camera for interpreting infrared warm imaging and AI face acknowledgment for separating swarm structures or doorways more effective, wherein the gadget catches a guest's temperature and furthermore checks whether the person in question is wearing a face veil or not.

4. The method of claim 1, wherein a chatbot is executed to provide an online discussion to a client about one or the other content or voice shows on a user interface, wherein the user interface is installed on a user computing device.

5. The method of claim 1, wherein the chatbot promotes assisting the clients with a superior comprehension of their circumstance and gives a few clues to clients so the person is allowed to make legitimate strides, wherein the chatbot suggests that an individual having a positive COVID-19 test along with a person in close contact with the affirmed cases is suggested to be isolated for 14 days.

6. The method of claim 1, wherein one of an unmanned aerial vehicle (UAV) or a robot with the machine learning approach in light of the requirement for keeping social separation in swarms.

7. The method of claim 4, wherein the generated alert is shown in the user interface of the computing device so that any of the person having the user interface gets alert about the COVID-19.

8. The method of claim 1, wherein the alert is automatically generated and sent to registered computing device and the user interface.

9. The method of claim 1, wherein the region-grown operator performs connected-components analysis on gray-scale pixel values and the neighboring pixels are compared to a reference pixel value in each pixel of the image for the region-based segmentation.

10. The method of claim 1, wherein an individual is screened with a live stream on a dashboard which permits cautioning the experts if there should arise an occurrence of breaking of COVID-19 guidelines.
